(19) United States
(12) Patent Application Publication
(10) Pub. No.: US 2004/0186105 A1
(43) Pub. Date: Sep. 23, 2004

(54) Pharmaceutical composition exhibiting consistent drug release profile

(76) Inventors: Carl T. Allenspach, Chicago, IL (US); Sreekant R. Nadkarni, Gurnee, IL (US); Daryl A. Roston, Norman, OK (US); Brian R. Rohrs, Scotts, MI (US); Roxana F. Schlam, Highland Park, IL (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/647,501

(22) Filed: Aug. 25, 2003

Related U.S. Application Data
(60) Provisional application No. 60/407,212, filed on Aug. 30, 2002.
(60) Provisional application No. 60/479,584, filed on Jun. 18, 2003.

(51) Int. Cl. A61K 31/50; A61K 31/415; A61K 31/365; A61K 31/18

(52) U.S. Cl. 514/247; 514/406; 514/471; 514/602

(57) ABSTRACT

An orally deliverable pharmaceutical composition comprises a drug of low water solubility and a pregelatinized starch having low viscosity and/or exhibiting a multimodal particle size distribution. A process for preparing such a composition comprises a step of selecting a pregelatinized starch having low viscosity and/or exhibiting a multimodal particle size profile, and a step of admixing the selected pregelatinized starch with a drug of low water solubility.

### RELEASE PROFILE

This application claims priority of U.S. provisional application Serial No. 60/407,212 filed on Aug. 30, 2002 and U.S. provisional application Serial No. 60/479,584 filed on Jun. 18, 2003.

### FIELD OF THE INVENTION

The present invention relates to orally deliverable pharmaceutical compositions containing a drug, for example a selective cyclooxygenase-2 (COX-2) inhibitory drug of low water solubility, as an active ingredient, to processes for preparing such compositions, to methods of treatment of COX-2 mediated disorders comprising orally administering such compositions to a subject, and to use of such compositions in manufacture of medicaments.

### BACKGROUND OF THE INVENTION

During the process of seeking approval for and registering a pharmaceutical product with the Food and Drug Administration (FDA) in the U.S. and corresponding regulatory authorities in other countries, a particular candidate drug product, for example a tablet, must be shown to meet certain pre-established in vivo bioavailability and in vitro dissolution rate criteria. As a quality control measure, once such a drug product has received FDA or similar regulatory approval, samples drawn from batches of manufactured product must meet the dissolution rate criteria established during the regulatory approval process.

Typically, a drug manufacturer performs in-process or bulk finished product dissolution testing on a manufactured drug product to ensure that each batch of product meets established dissolution criteria; any drug product not meeting such criteria cannot be released to market and thus represents potentially wasted raw materials, labor, energy and resources. Therefore, from regulatory, production efficiency, financial and human resource perspectives, it is desirable that lot-to-lot, batch-to-batch and/or inter-tablet dissolution rate differences, and/or any other dissolution rate differences potentially present in a given manufacturing campaign, are negligible or small enough so as not to result in failure of product to meet pre-established dissolution rate criteria.

Furthermore, it is desirable also from safety and efficacy standpoints that lot-to-lot, batch-to-batch and/or inter-tablet dissolution rate differences are minimal. Where substantial variability in drug dissolution exists, some tablets can dissolve very quickly while others dissolve more slowly. Those tablets exhibiting increased dissolution rate can provide more rapid in vivo release, which in turn can lead to higher blood levels of the drug shortly after administration, with potentially increased risk for undesirable side-effects. Conversely, those tablets exhibiting decreased dissolution rate can provide less rapid in vivo release, which in turn can lead to lower blood levels of the drug shortly after administration, with potentially increased risk for reduced therapeutic response.

The compound 4-(5-methyl-3-phenyl-4-isoxazolyl)benzenesulfonamide, also referred to herein as valdecoxib, was disclosed in U.S. Pat. No. 5,633,272 to Talley et al. together with processes for preparing this and related compounds. Valdecoxib has the structure:

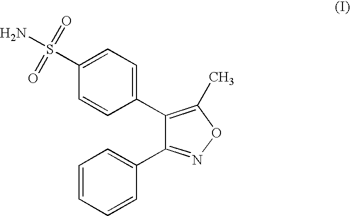

The compounds reported in above-cited U.S. Pat. No. 5,633,272, including valdecoxib, are disclosed therein as useful anti-inflammatory, analgesic and antipyretic drugs having a high degree of selectivity for inhibition of cyclooxygenase-2 (COX-2) over cyclooxygenase-1 (COX-1). Above-cited U.S. Pat. No. 5,633,272 also contains general references to formulations for the administration of such compounds, including orally deliverable dosage forms such as tablets and capsules.

European Patent Application No. 0 863 134 discloses orally deliverable compositions comprising a selective COX-2 inhibitory drug, specifically 2-(3,5-difluorophenyl)-3-(4-methyl-sulfonyl)phenyl)-2-cyclopenten-1-one, in combination with excipient ingredients including microcrystalline cellulose, lactose monohydrate, hydroxypropylcellulose, croscarmellose sodium and magnesium stearate.

International Patent Publication No. WO 00/32189 discloses orally deliverable compositions comprising a selective COX-2 inhibitory drug, specifically celecoxib, in combination with excipient ingredients selected from extensive lists of suitable diluents, disintegrants, binding agents, wetting agents, lubricants, etc.

International Patent Publication No. WO 01/41762 describes orally deliverable pharmaceutical compositions containing, inter alia, valdecoxib and pregelatinized starch (e.g., National Starch 1500). Pregelatinized starch is a commonly used excipient in pharmaceutical dosage forms and is generally employed as a diluent, disintegrant, and/or binder.

We have now discovered that pharmaceutical dosage forms (e.g., tablets) comprising a drug of low water solubility (e.g., valdecoxib) and pharmaceutical grade pregelatinized starch can exhibit the undesirable attribute of drug dissolution rate variability. As indicated above, drug dissolution rate variability is particularly undesirable as it can lead to side effects, lack of therapeutic response in some patients, and/or production inefficiencies.

If orally deliverable pharmaceutical dosage forms comprising a drug of low water solubility (e.g., valdecoxib) and pregelatinized starch could be prepared exhibiting the desirable attribute of improved dissolution rate uniformity, a significant advance in the safety, efficacy and production efficiency of many pharmaceutical dosage forms would be realized.

### SUMMARY OF THE INVENTION

There is now provided an orally deliverable pharmaceutical composition comprising a drug of low water solubility and a pregelatinized starch having low viscosity and/or exhibiting a multimodal particle size distribution. The composition is illustrated herein with reference to a selective COX-2 inhibitory drug of low water solubility, for example valdecoxib.

Such a composition has been found to exhibit a surprising and unexpected increase in drug dissolution rate consistency by comparison with otherwise similar compositions comprising a pregelatinized starch other than that specified immediately above.

There is further provided a method for improving drug dissolution rate consistency among pharmaceutical tablets prepared within a single manufacturing campaign, such tablets comprising a drug, illustratively a selective COX-2 inhibitory drug, of low water solubility and pregelatinized starch. The method comprises a step of selecting a pregelatinized starch having low viscosity and/or exhibiting a multi-modal particle size distribution.

There is still further provided a method of treating and/or preventing a COX-2 mediated condition or disorder in a subject, the method comprising administering to the subject a therapeutically and/or prophylactically effective amount of a composition of the invention.

### BRIEF DESCRIPTION OF THE DRAWINGS

FIG. 1 shows rheology in the form of a graph of shear stress versus shear rate, determined according to Test I described hereinbelow, for pregelatinized starch samples prepared in six different manufacturing lots.

FIG. 2 shows bimodal particle size distribution exhibited by pregelatinized starch sampled from Lot H, as measured by laser diffraction according to Example 4.

FIG. 3 shows unimodal particle size distribution exhibited by pregelatinized starch sampled from Lot K, as measured by laser diffraction according to Example 4.

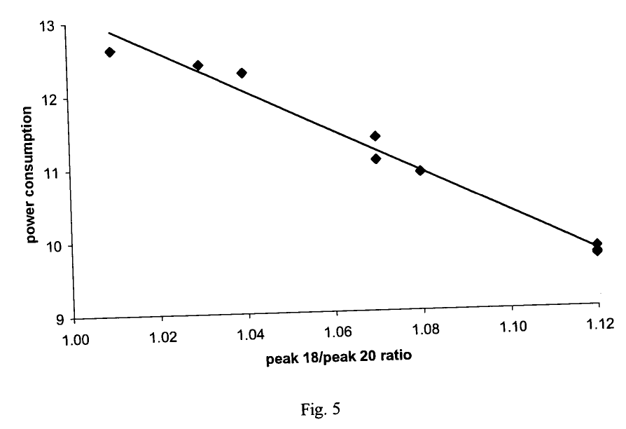

### DETAILED DESCRIPTION OF THE INVENTION

The present composition comprises a drug, illustratively a selective COX-2 inhibitory drug, of low water solubility, and a pregelatinized starch having low viscosity and/or exhibiting a multimodal particle size distribution. Optionally one or more additional pharmaceutically acceptable excipients are present in the composition. Preferably, the composition is in a form of orally deliverable tablets, capsules or granules.

A “drug of low water solubility” or a “poorly water solubility drug” herein means a drug having solubility in water, measured at 37° C., not greater than about 10 mg/ml, and preferably not greater than about 1 mg/ml. It is contemplated that compositions of the invention are especially advantageous for drugs having solubility in water, measured for example at ambient temperature (about 20° C. to about 25° C.) and/or at body temperature (about 37° C.), not greater than about 0.1 mg/ml. Solubility in water for many drugs can be readily determined from standard pharmaceutical reference books, for example The Merck Index, 13th ed., 2001 (published by Merck & Co., Inc., Rahway, N.J.); the United States Pharmacopeia, 24th ed. (USP 24), 2000; The Extra Pharmacopoeia, 29th ed., 1989 (published by Pharmaceutical Press, London); and the Physicians Desk Reference (PDR), 2001 ed. (published by Medical Economics Co., Montvale, N.J.). Unless the context demends otherwise, “drugs” herein include prodrugs, salts and active metabolites of drugs.

For example, individual drugs of low water solubility as defined herein include those drugs categorized as “slightly soluble”, “very slightly soluble”, “practically insoluble” and “insoluble” in USP 24, pp. 2254-2298; and those drugs categorized as requiring 100 nml or more of water to dissolve 1 g of the drug, as listed in USP 24, pp. 2299-2304.

Illustratively, suitable drugs of low water solubility include, without limitation, drugs from the following classes: abortifacients, ACE inhibitors, α- and β-adrenergic agonists, α- and β-adrenergic blockers, adrenocortical suppressants, adrenocorticotropic hormones, alcohol deterrents, aldose reductase inhibitors, aldosterone antagonists, anabolics, analgesics (including narcotic and non-narcotic analgesics), androgens, angiotensin II receptor antagonists, anorexics, antacids, anthelninthics, antiacne agents, antiallergics, antialopecia agents, antiamebics, antiandrogens, antianginal agents, antiarrhythmics, antiarteriosclerotics, antiarthritic/antirheumatic agents (including selective COX-2 inhibitors), antiasthmatics, antibacterials, antibacterial adjuncts, anticholinergics, anticoagulants, anticonvulsants, antidepressants, antidiabetics, antidiarrheal agents, antidiuretics, antidotes to poison, antidyskinetics, antieczematics, antiemetics, antiestrogens, antifibrotics, antiflatulents, antifungals, antiglaucoma agents, antigonadotropins, antigout agents, antihistaminics, antihyperactives, antihyperlipoproteinemics, antihyperphosphatemics, antihypertensives, antihyperthyroid agents, antihypotensives, antihypothyroid agents, anti-inflammatories, antimalarials, antimanics, antimethemoglobinemics, antimigraine agents, antimuscarinics, antimycobacterials, antineoplastic agents and adjuncts, antineutropenics, antiosteoporotics, antipagetics, antiparkinsonian agents, antipheochromocytoma agents, antipneumocystis agents, antiprostatic hypertrophy agents, antiprotozoals, antipruritics, antipsoriatics, antipsychotics, antipyretics, antirickettsials, antiseborrheics, antiseptics/disinfectants, antispasmodics, antisyphylitics, antithrombocythemics, antithrombotics, antitussives, antiulceratives, antiurolithics, antivenins, antiviral agents, anxiolytics, aromatase inhibitors, astringents, benzodiazepine antagonists, bone resorption inhibitors, bradycardic agents, bradykinin antagonists, bronchodilators, calcium channel blockers, calcium regulators, carbonic anhydrase inhibitors, cardiotonics, CCK antagonists, chelating agents, cholelitholytic agents, choleretics, cholinergics, cholinesterase inhibitors, cholinesterase reactivators, CNS stimulants, contraceptives, debriding agents, decongestants, depigmentors, dermatitis herpetiformis suppressants, digestive aids, diuretics, dopamine receptor agonists, dopamine receptor antagonists, ectoparasiticides, emetics, enkephalinase inhibitors, enzymes, enzyme cofactors, estrogens, expectorants, fibrinogen receptor antagonists, fluoride supplements, gastric and pancreatic secretion stimulants, gastric cytoprotectants, gastric proton pump inhibitors, gastric secretion inhibitors, gastroprokinetics, glucocorticoids, α-glucosidase inhibitors, gonad-stimulating principles, growth hormone inhibitors, growth hormone releasing factors, growth stimulants, hematinics, hematopoietics, hemolytics, hemostatics, heparin antagonists, hepatic enzyme inducers, hepatoprotectants, histamine H₂ receptor antagonists, HIV protease inhibitors, HMG CoA reductase inhibitors, immunomodulators, immunosuppressants, insulin sensitizers, ion exchange resins, keratolytics, lactation stimulating hormones, laxatives/cathartics, leukotriene antagonists, LH-RH agonists, lipotropics, 5-lipoxygenase inhibitors, lupus erythematosus suppressants, matrix metalloproteinase inhibitors, mineralocorticoids, miotics, monoamine oxidase inhibitors, mucolytics, muscle relaxants, mydriatics, narcotic antagonists, neuroprotectives, nootropics, ovarian hormones, oxytocics, pepsin inhibitors, pigmentation agents, plasma volume expanders, potassium channel activators/openers, progestogens, prolactin inhibitors, prostaglandins, protease inhibitors, radio-pharmaceuticals, 5α-reductase inhibitors, respiratory stimulants, reverse transcriptase inhibitors, sedatives/hypnotics, serenics, serotonin noradrenaline reuptake inhibitors, serotonin receptor agonists, serotonin receptor antagonists, serotonin uptake inhibitors, somatostatin analogs, thrombolytics, thromboxane A₂ receptor antagonists, thyroid hormones, thyrotropic hormones, tocolytics, topoisomerase I and II inhibitors, uricosurics, vasomodulators including vasodilators and vasoconstrictors, vasoprotectants, xanthine oxidase inhibitors, and combinations thereof.

Non-limiting illustrative examples of suitable drugs of low water solubility include acetohexamide, acetylsalicylic acid, alclofenac, allopurinol, atropine, benzthiazide, carprofen, celecoxib, chlordiazepoxide, chlorpromazine, clonidine, codeine, codeine phosphate, codeine sulfate, deracoxib, diacerein, diclofenac, diltiazem, estradiol, etodolac, etoposide, etoricoxib, fenbufen, fenclofenac, fenprofen, fentiazac, flurbiprofen, griseofulvin, haloperidol, ibuprofen, indomethacin, indoprofen, ketoprofen, lorazepam, medroxyprogesterone acetate, megestrol, methoxsalen, methylprednisone, morphine, morphine sulfate, naproxen, nicergoline, nifedipine, niflumic, oxaprozin, oxazepam, oxyphenbutazone, paclitaxel, phenindione, phenobarbital, piroxicam, pirprofen, prednisolone, prednisone, procaine, progesterone, pyrimethamine, rofecoxib, sulfadiazine, sulfamerazine, sulfisoxazole, sulindac, suprofen, temazepam, tiaprofenic acid, tilomisole, tolmetic, valdecoxib, etc. The invention is illustrated herein with particular reference to valdecoxib.

The amount of drug to be incorporated in a composition of the invention can be selected according to known principles of pharmacy. A therapeutically and/or prophylactically effective amount of drug is specifically contemplated. The term “therapeutically and/or prophylactically effective amount” as used herein refers to an amount of drug present in a unit dose of the composition, for example a single tablet or capsule, that is sufficient to elicit a required or desired therapeutic and/or prophylactic response.

What constitutes a therapeutically and/or prophylactically effective amount depends greatly on the particular drug. In most cases, however, the drug is present in a composition of the invention in a concentration of at least about 0.01%, preferably at least about 0.1%, more preferably at least about 1%, still more preferably at least about 2.5%, and even more preferably at least about 5%, by weight. Unless otherwise indicated, all concentrations given herein are on a weight/weight basis.

In a preferred embodiment, the drug is a selective COX-2 inhibitory drug. A preferred selective COX-2 inhibitory drug useful herein, or to which a salt or prodrug useful herein is converted in vivo, is a compound of formula (II):

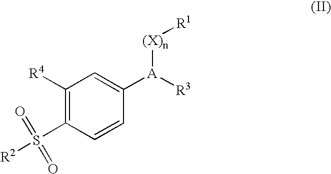

wherein:

A is a substituent selected from partially unsaturated or unsaturated heterocyclyl and partially unsaturated or unsaturated carbocyclic rings, preferably a heterocyclyl group selected from pyrazolyl, furanonyl, isoxazolyl, pyridinyl, cyclopentenonyl and pyridazinonyl groups;

X is O, S or CH₂;

n is 0 or 1;

R¹ is at least one substituent selected from heterocyclyl, cycloalkyl, cycloalkenyl and aryl, and is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R² is methyl, amino or aminocarbonylalkyl;

R³ is one or more radicals selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl, R³ being optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; and

R⁴ is selected from hydrido and halo.

Compositions of the invention are especially useful for selective COX-2 inhibitory drugs of formula (III):

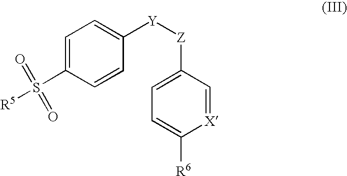

where R⁵ is a methyl or amino group, R⁶ is hydrogen or a C₁₋₄ alkyl or alkoxy group, X′ is N or CR⁷ where R⁷ is hydrogen or halogen, and Y and Z are independently carbon or nitrogen atoms defining adjacent atoms of a five- to six-membered ring that is optionally substituted at one or more positions with oxo, halo, methyl or halomethyl groups, or an isomer, tautomer, pharmaceutically-acceptable salt or prodrug thereof. Preferred such five- to six-membered rings are cyclopentenone, furanone, methylpyrazole, isoxazole and pyridine rings substituted at no more than one position.

Illustratively, compositions of the invention are suitable for celecoxib, deracoxib, valdecoxib, rofecoxib, etoricoxib, 2-(3,5-difluorophenyl)-3-[4-(methylsulfonyl)phenyl]-2-cyclopenten-1-one, 2-(3,4-difluorophenyl)-4-(3-hydroxy-3-methyl-1-butoxy)-5-[4-(methylsulfonyl)phenyl]-3-(2H)-pyridazinone, pharmaceutically acceptable salts and prodrugs thereof, so long as these meet the solubility criteria established herein.

Compositions of the invention are also useful for selective COX-2 inhibitory drugs of formula (IV):

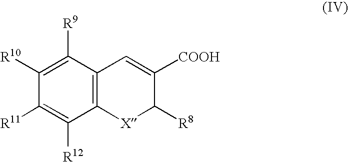

where X″ is O, S or N-lower alkyl; R⁸ is lower haloalkyl; R⁹ is hydrogen or halogen; R¹⁰ is hydrogen, halogen, lower alkyl, lower alkoxy or haloalkoxy, lower aralkylcarbonyl, lower dialkylaminosulfonyl, lower alkylaminosulfonyl, lower aralkylaminosulfonyl, lower heteroaralkylaminosulfonyl, or 5- or 6-membered nitrogen-containing heterocyclosulfonyl; and R¹¹ and R¹² are independently hydrogen, halogen, lower alkyl, lower alkoxy, or aryl; and for pharmaceutically acceptable salts thereof.

In a particularly preferred embodiment, the drug of low water solubility is also a drug such as valdecoxib having a relatively low daily dose requirement, for example a requirement for not greater than about 100 mg/day.

For many drugs including valdecoxib, particle size reduction can lead to improved bioavailability when the drug is formulated in a composition of the invention. Illustratively in the case of valdecoxib, the D₉₀ particle size is preferably less than about 75 μm, for example about 1 to about 70 μm, about 1 to about 40 μm or about 1 to about 30 μm. The term “D₉₀ particle size” in relation to a drug sample means a diameter, in the longest dimension of the particles, such that 90% by weight of all particles present in the sample are smaller than that diameter. In addition or alternatively, valdecoxib used in a composition of the invention preferably has a weight average particle size of about 1 to about 10 μm, more preferably about 5 to about 7 μm. Any suitable milling, grinding or micronizing method can be used for particle size reduction to the desired range.

In a preferred valdecoxib composition of the invention, each unit dose preferably comprises valdecoxib in an amount of about 1 to about 100 mg, more preferably about 2 to about 60 mg, and more preferably about 5 to about 40 mg, for example about 5 mg, about 10 mg, about 20 mg or about 40 mg.

In addition to at least one drug as described above, a composition of the invention comprises a pregelatinized starch having low viscosity and/or exhibiting a multi-modal particle size distribution.

Pregelatinized starch is starch that has been physically and/or chemically processed to rupture some or all starch granules. Such processing tends to render starch granules flowable and directly compressible. The term “pregelatinized starch” herein includes starches described elsewhere as partially pregelatinized starch. Illustratively, pregelatinized starch contains about 2% to about 10%, for example about 5%, free amylose, about 10% to about 20%, for example about 15%, free amylopectin, and about 60% to about 90%, for example about 80%, unmodified starch. Pregelatinized starch is commonly used in oral capsule and tablet formulations as a binder, diluent and/or disintegrant. Suitable pregelatinized starch can be derived from any botanic origin, for example corn (maize), wheat, cassaya, potato, etc., but preferably from corn. Non-limiting examples of commercially available pregelatinized corn starches from which a low viscosity pregelatinized starch useful in the invention can be selected include National Starch 78-1551 and Starch 1500 of Colorcon.

A composition of the invention comprises not less than about 0.1%, preferably not less than about 1%, more preferably not less than about 2.5%, and most preferably not less than about 5%, pregelatinized starch, by weight. Illustratively, a composition of the invention comprises about 1% to about 50%, preferably about 2.5% to about 30%, and more preferably about 5% to about 25%, pregelatinized starch, by weight.

In a first embodiment, the pregelatinized starch is of low viscosity. Whether a sample of pregelatinized starch is one having “low viscosity” as that term is used herein can illustratively be determined according to Test I.

Test I

A. A test sample of a pregelatinized starch is selected or provided.

B. A 1 g aliquot of the test sample is placed in a 20 ml glass scintillation vial at room temperature.

C. Water at room temperature, in an amount of 10 ml, is added to the scintillation vial to form a mixture with the starch.

D. The mixture is vortexed for 1 minute and then stirred for 2 hours at 500 rpm on an orbital stirrer.

E. A 2 g sample of the mixture is then drawn and placed in a viscometer sensor (illustratively a Haake CV 100 rotational viscometer with a PK30-40 sensor).

F. Shear stress, increasing from 0 to 100 s⁻¹ over a period of 3 minutes, is applied to the sample in the viscometer and dynamic viscosity is measured.

G. If the sample exhibits, at a shear rate of 20 s⁻¹, a shear stress of not more than about 1 Pa, preferably not more than about 0.75 Pa, the pregelatinized starch is deemed have “low viscosity” as required in the present embodiment.

It will be understood that small variations can be made in the conditions under which Test I is run without significantly affecting the outcome.

It is preferred according to the present embodiment that the pregelatinized starch additionally exhibit, in Test I, a shear stress of not more than about 2 Pa, more preferably not more than about 1.5 Pa, at a shear rate at 60 s⁻¹.

For example, a low viscosity pregelatinized starch can be selected that exhibits, in Test I, a shear stress of not more than about 1 Pa at 20 s⁻¹, not more than about 2 Pa at 60 s⁻¹, and not more than about 3 Pa at 100 s^{-1.}

Alternatively, a low viscosity pregelatinized starch can be selected that exhibits, in Test I, a shear stress of not more than about 0.75 Pa at 20 s⁻¹, not more than about 1.5 Pa at 60 s⁻¹, and not more than about 2.5 Pa at 100 s⁻¹.

Alternatively, a low viscosity pregelatinized starch can be selected that exhibits, in Test I, a shear stress of not more than about 0.5 Pa at 20 s⁻¹, not more than about 1 Pa at 60 s⁻¹, and not more than about 1.5 Pa at 100 s⁻¹.

In a second embodiment, the pregelatinized starch is one exhibiting a multimodal particle size distribution. The term “multimodal” herein embraces any particle size distribution having more than one maximum, i.e., including bimodal and trimodal but not unimodal distributions.

Whether a sample of pregelatinized starch exhibits a multimodal particle size distribution can be determined using any suitable analytical particle size technique. Illustratively, particle size distribution of dry pregelatinized starch can be determined by laser diffraction, for example using a Sympatec HELOS in Fraunhofer optical mode and using a 500 mm lens.

Compositions of the invention are orally deliverable and can be in a form, for example, of tablets, caplets, hard or soft capsules, lozenges, cachets, dispensable powder blends, granules, etc. Once a pregelatinized starch having low viscosity and/or multimodal particle size distribution has been selected, such a composition can be prepared by any suitable method of pharmacy that includes a step of bringing into association the drug, the pregelatinized starch and any other desired excipient(s). In general, a composition is prepared by uniformly and intimately admixing the drug, the pregelatinized starch and the optional additional excipients with a liquid or finely divided solid diluent, and then, if capsules or tablets are required, encapsulating or tableting the resulting blend. For example, a tablet can be prepared by compressing or molding a powder or granules of such a blend, optionally together with one or more additional excipients. Compressed tablets can illustratively be prepared by compressing, in a suitable machine, a free-flowing composition, such as a powder or granules, comprising the admixture of the drug and the pregelatinized starch optionally mixed with one or more diluents, disintegrants, binding agents and/or lubricants. Molded tablets can illustratively be prepared by molding, in a suitable machine, a powder comprising the admixture of the drug and the pregelatinized starch optionally mixed with one or more excipients, moistened with a liquid diluent.

A composition of the invention can be in standard-release, immediate-release, rapid-onset, sustained-release or dual-release form. Where the composition comprises valdecoxib, it is preferably an immediate-release composition that releases at least about 75%, more preferably at least about 80%, of the valdecoxib within about 45 minutes, as measured in a standard in vitro dissolution assay. Especially preferred valdecoxib compositions of the invention release in vitro at least about 50% of the valdecoxib within about 15 minutes, and/or at least about 60% of the valdecoxib within about 30 minutes.

An illustrative standard in vitro dissolution assay is performed according to USP 24 using Apparatus 2, in a dissolution medium of 1000 ml of 1% weight/volume sodium dodecyl sulfate (SDS) maintained at 37° C., at a paddle speed of 75 rpm. Alternatively, USP 24 Apparatus 1 can be used.

In addition to at least one drug and a pregelatinized starch selected as described above, a composition of the invention optionally comprises one or more additional pharmaceutically acceptable diluents as excipients. Suitable diluents illustratively include, either individually or in combination, lactose, including anhydrous lactose and lactose monohydrate; mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of α- and amorphous cellulose (e.g., Rexcel™) and powdered cellulose; calcium carbonate; glycine; bentonite; polyvinylpyrrolidone; and the like. Such diluents, if present, constitute in total about 5% to about 99%, preferably about 10% to about 85%, and more preferably about 20% to about 80%, by weight of the composition. The diluent or diluents selected preferably exhibit suitable flow properties and, where tablets are desired, compressibility.

Lactose and microcrystalline cellulose, either individually or in combination, are preferred diluents. Both these diluents are chemically compatible with valdecoxib. The use of extragranular microcrystalline cellulose (that is, microcrystalline cellulose added to a wet granulated composition after a drying step) can improve hardness and/or disintegration time of tablets. Lactose, especially lactose monohydrate, is particularly preferred. Lactose typically provides compositions having suitable release rates of valdecoxib, stability, pre-compression flowability, and/or drying properties at a relatively low diluent cost. It provides a high-density substrate that aids densification during wet granulation and therefore improves blend flow properties.

A composition of the invention optionally further comprises one or more pharmaceutically acceptable disintegrants as excipients, particularly for tablet formulations. Suitable disintegrants include, either individually or in combination, starches in addition to the pregelatinized starch already present, such as starch glycolate (e.g., Explotab™ of PenWest); clays (e.g., Veegum™ HV); cellulose-based disintegrants such as purified cellulose, microcrystalline cellulose, methylcellulose, carmellose, carmellose sodium and croscarmellose sodium (e.g., Ac-Di-Sol™ of FMC); alginates; crospovidone; and gums such as agar, guar, locust bean, karaya, pectin and tragacanth gums.

A disintegrant can be added at any suitable step during preparation of the composition, particularly prior to granulation or during a blending or lubrication step prior to compression. One or more disintegrants, if present, constitute in total about 0.2% to about 30%, preferably about 0.2% to about 10%, and more preferably about 0.2% to about 5%, by weight of the composition.

Croscarmellose sodium is a preferred disintegrant for tablet or capsule formulations, and, if present, preferably constitutes about 0.2% to about 10%, more preferably about 0.2% to about 7%, and still more preferably about 0.2% to about 5%, by weight of the composition. Croscarmellose sodium confers superior intragranular disintegration capabilities to granulated compositions.

A composition of the invention optionally further comprises one or more pharmaceutically acceptable binding agents or adhesives as excipients, particularly where the composition is in the form of a tablet. Such binding agents and adhesives preferably impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; celluloses including, but not limited to, methylcellulose, carmellose sodium (e.g., Tylose™), hydroxypropylmethylcellulose (HPMC), hydroxypropylcellulose (e.g., Klucel™) and ethylcellulose (e.g., Ethocel™); alginic acid and salts of alginic acid; magnesium aluminum silicate; polyethylene glycol (PEG); guar gum; polysaccharide acids; bentonites; polyvinylpyrrolidone (povidone or PVP), for example povidone K-15, K-30 and K-29/32; and polymethacrylates. One or more binding agents and/or adhesives, if present, constitute in total about 0.5% to about 25%, preferably about 0.75% to about 15%, and more preferably about 1% to about 10%, by weight of the composition.

A composition of the invention optionally further comprises one or more pharmaceutically acceptable wetting agents as excipients. Such wetting agents are preferably selected to maintain the drug in close association with water, a condition that is believed to improve bioavailability of the composition.

Non-limiting examples of surfactants that can be used as wetting agents include quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides (e.g., Labrasol™ of Gattefosse), polyoxyethylene (35) castor oil and polyoxyethylene (40) hydrogenated castor oil; polyoxyethylene alkyl ethers, for example polyoxyethylene (20) cetostearyl ether; polyoxyethylene fatty acid esters, for example polyoxyethylene (40) stearate; polyoxyethylene sorbitan esters, for example polysorbate 20 and polysorbate 80 (e.g., Tween™ 80); propylene glycol fatty acid esters, for example propylene glycol laurate (e.g., Lauroglycol™ of Gattefosse); sodium lauryl sulfate; fatty acids and salts thereof, for example oleic acid, sodium oleate and triethanolamine oleate; glyceryl fatty acid esters, for example glyceryl monostearate; sorbitan esters, for example sorbitan monolaurate, sorbitan monooleate, sorbitan monopalmitate and sorbitan monostearate; tyloxapol; and mixtures thereof. One or more wetting agents, if present, constitute in total about 0.25% to about 15%, preferably about 0.4% to about 10%, and more preferably about 0.5% to about 5%, by weight of the composition.

Wetting agents that are anionic surfactants are preferred. Sodium lauryl sulfate is a particularly preferred wetting agent. Sodium lauryl sulfate, if present, constitutes about 0.25% to about 7%, more preferably about 0.4% to about 4%, and still more preferably about 0.5% to about 2%, by weight of the composition.

A composition of the invention optionally further comprises one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium, calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEGs (e.g. Carbowax™ 4000 and Carbowax™ 6000); sodium oleate; sodium lauryl sulfate; and magnesium lauryl sulfate. One or more lubricants, if present, constitute in total about 0.1% to about 10%, preferably about 0.2% to about 8%, and more preferably about 0.25% to about 5%, by weight of the composition.

Magnesium stearate is a preferred lubricant used, for example, to reduce friction between the equipment and granulated mixture during compression of tablet formulations.

Suitable anti-adherents include talc, cornstarch, DL-leucine, sodium lauryl sulfate and metallic stearates. Talc is a preferred anti-adherent or glidant used, for example, to reduce formulation sticking to equipment surfaces and also to reduce static in the blend. Talc, if present, constitutes about 0.1% to about 10%, more preferably about 0.25% to about 5%, and still more preferably about 0.5% to about 2%, by weight of the composition.

Other excipients such as colorants, flavors and sweeteners are known in the pharmaceutical art and can be used in compositions of the present invention. Tablets can be coated, for example with an immediate-release, delayed-release or enteric coating, or uncoated. Compositions of the invention can further comprise, for example, buffering agents.

Although compositions of the invention can be prepared, for example, by direct encapsulation or direct compression, they are preferably wet granulated prior to encapsulation or tableting. Wet granulation, among other effects, densifies milled compositions resulting in improved flow properties, improved compression characteristics and easier metering or weight dispensing of a blend for encapsulation or tableting. Desired bulk density of the resulting granules when poured or tapped is normally about 0.3 to about 1.0 g/ml, for example about 0.5 to about 0.9 g/ml.

To prepare tablets by compression, the granulated blend in an amount sufficient to make a uniform batch of tablets can be processed in a conventional production scale tableting machine at normal compression pressure (for example, applying a force of about 1 to about 50 kN in a typical tableting die). The resulting tablet hardness should be convenient with respect to handling, manufacture, storage and ingestion; however a minimum hardness of about 4 kP, preferably about 5 kP and more preferably about 6 kP, is desirable to avoid excessive friability, and a maximum hardness of about 18 kP, preferably about 15 kP and more preferably about 12 kP, is desirable to avoid subsequent difficulty in hydrating the tablet when exposed to gastric fluid. When hardness is in an acceptable range, tablet friability is typically less than about 1.0%, preferably less than about 0.8% and more preferably less than about 0.5%, in a standard test.

Excipients, in particular a disintegrant, for immediate release capsule and tablet compositions of the invention are preferably selected to provide a disintegration time of less than about 30 minutes, preferably less than about 25 minutes, more preferably less than about 20 minutes, and still more preferably less than about 15 minutes, in a standard in vitro disintegration assay.

Where a composition of the invention comprises a selective COX-2 inhibitory drug, it is useful in treatment and prevention of a very wide range of disorders mediated by COX-2, including but not restricted to disorders characterized by inflammation, pain and/or fever. Such compositions are especially useful as anti-inflammatory agents, such as in treatment of arthritis, with the additional benefit of having significantly less harmful side effects than compositions of conventional NSAIDs that lack selectivity for COX-2 over COX-1. In particular, compositions of the invention have reduced potential for gastrointestinal toxicity and gastrointestinal irritation, including upper gastrointestinal ulceration and bleeding, by comparison with compositions of conventional NSAIDs. Thus compositions of the invention are particularly useful as an alternative to conventional NSAIDs where such NSAIDs are contraindicated, for example in patients with peptic ulcers, gastritis, regional enteritis, ulcerative colitis, diverticulitis or with a recurrent history of gastrointestinal lesions; gastrointestinal bleeding, coagulation disorders including anemia such as hypoprothrombinemia, hemophilia or other bleeding problems; kidney disease; or in patients prior to surgery or patients taking anticoagulants.

Contemplated compositions are useful to treat a variety of arthritic disorders, including but not limited to rheumatoid arthritis, spondyloarthropathies, gouty arthritis, osteoarthritis, systemic lupus erythematosus and juvenile arthritis.

Such compositions are useful in treatment of asthma, bronchitis, menstrual cramps, preterm labor, tendonitis, bursitis, allergic neuritis, cytomegalovirus infection, apoptosis including HIV-induced apoptosis, lumbago, liver disease including hepatitis, skin-related conditions such as psoriasis, eczema, acne, burns, dermatitis and ultraviolet radiation damage including sunburn, and post-operative inflammation including that following ophthalmic surgery such as cataract surgery or refractive surgery.

Such compositions are useful to treat gastrointestinal conditions such as inflammatory bowel disease, Crohn's disease, gastritis, irritable bowel syndrome and ulcerative colitis.

Such compositions are useful in treating inflammation in such diseases as migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

Such compositions are useful in treatment of ophthalmic disorders, including without limitation inflammatory disorders such as endophthalmitis, episcleritis, retinitis, iriditis, cyclitis, choroiditis, keratitis, conjunctivitis and blepharitis, inflammatory disorders of more than one part of the eye, e.g., retinochoroiditis, iridocyclitis, iridocyclochoroiditis (also known as uveitis), keratoconjunctivitis, blepharoconjunctivitis, etc.; other COX-2 mediated retinopathies including diabetic retinopathy; ocular photophobia; acute trauma of any tissue of the eye including postsurgical trauma, e.g., following cataract or corneal transplant surgery; postsurgical ocular inflammation; intraoperative miosis; corneal graft rejection; ocular, for example retinal, neovascularization including that following injury or infection; macular degeneration; cystoid macular edema; retrolental fibroplasia; neovascular glaucoma; and ocular pain.

Such compositions are useful in treatment of pulmonary inflammation, such as that associated with viral infections and cystic fibrosis, and in bone resorption such as that associated with osteoporosis.

Such compositions are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such compositions are useful in treatment of allergic rhinitis, respiratory distress syndrome, endotoxin shock syndrome and liver disease.

Such compositions are useful in treatment of pain, including but not limited to postoperative pain, dental pain, muscular pain, and pain resulting from cancer. For example, such compositions are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, burns, and trauma following surgical and dental procedures.

Such compositions are useful for treating and preventing inflammation-related cardiovascular disorders, including vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Such compositions are useful in treatment of angiogenesis-related disorders in a subject, for example to inhibit tumor angiogenesis. Such compositions are useful in treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemangiomas, angiofibroma of the nasopharynx and avascular necrosis of bone; and disorders of the female reproductive system such as endometriosis.

Such compositions are useful in prevention and treatment of benign and malignant tumors and neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. Such compositions can also be used to treat fibrosis that occurs with radiation therapy. Such compositions can be used to treat subjects having adenomatous polyps, including those with familial adenomatous polyposis (FAP). Additionally, such compositions can be used to prevent polyps from forming in subjects at risk of FAP.

Such compositions inhibit prostanoid-induced smooth muscle contraction by inhibiting synthesis of contractile prostanoids and hence can be of use in treatment of dysmenorrhea, premature labor, asthma and eosinophil-related disorders. They also can be of use for decreasing bone loss particularly in postmenopausal women (i.e., treatment of osteoporosis), and for treatment of glaucoma.

Preferred uses for compositions of the invention are for treatment of rheumatoid arthritis and osteoarthritis, for pain management generally (particularly post-oral surgery pain, post-general surgery pain, post-orthopedic surgery pain, and acute flares of osteoarthritis), for treatment of Alzheimer's disease, and for colon cancer chemoprevention.

Besides being useful for human treatment, compositions of the invention are useful for veterinary treatment of companion animals, exotic animals, farm animals, and the like, particularly mammals. More particularly, compositions of the invention are useful for treatment of COX-2 mediated disorders in horses, dogs and cats.

### EXAMPLES

The following examples are provided for illustrative purposes only and are not to be construed as limitations.

### Example 1

Samples of Starch 1500, a pregelatinized starch supplied by Colorcon, which had been manufactured in 11 different lots (Lots A-K) were obtained. Starch from each lot was individually used in preparing batches of valdecoxib immediate-release tablets having the composition shown in Table 1, according to the procedure described below.

TABLE 1
Composition of valdecoxib tablets prepared with
various lots of starch

| Component | Function | Amount (mg) |
|---|---|---|
| valdecoxib, micronized | active ingredient | 10 |
| lactose monohydrate NF | primary diluent | 103 |
| microcrystalline cellulose NF | secondary diluent | |
| intragranular | | 30 |
| extragranular | | 30 |
| pregelatinized starch NF | binding agent | 20 |
| (Starch 1500) | | |
| croscarmellose sodium NF | disintegrant | |
| intragranular | | 3 |
| extragranular | | 3 |
| magnesium stearate | lubricant | 1 |
| Total tablet weight | | 200 |

Valdecoxib (previously micronized), lactose monohydrate NF, pregelatinized starch NF, croscarmellose sodium NF and microcrystalline cellulose NF were transferred into a high shear granulator bowl and were mixed at high impeller and chopper speeds to form a premix.

The premix was granulated with purified water while mixing at high impeller and chopper speeds to form a wet mass. The mixing was stopped when a target water addition amount was reached; no mixing was done after water addition was complete. The wet mass was discharged from the granulator bowl and de-lumped by passing through a screening mill and resulting wet granules were collected into a fluid bed dryer bowl.

The wet granules were then dried in a fluid bed dryer at an inlet air temperature of 70° C. until a loss on drying (LOD) of the granules of 2.0% was attained. The dried granules were sized by passing through a cutting mill using a 20 mesh screen. The dry, sized granules were then loaded into a V-blender and blended with extra-granular microcrystalline cellulose NF and croscarmellose sodium NF to form a uniform blend. Magnesium stearate was then added and blended to form a lubricated blend.

The lubricated blend was charged into a tablet press hopper and compressed into core tablets at hardnesses of 6 kP, 9 kP and 12 kP.

### Example 2

Eleven batches (1-11) of tablets prepared as in Example 1 (using starch from lots A-K respectively) were evaluated in an in vitro dissolution test according to the following procedure.

Tablets were individually placed in 1000 ml of 1% SDS maintained at 37° C., which was stirred at 75 rpm using USP 24 Apparatus 1. Drug concentration in the SDS was measured 45 minutes after the start of the test.

Prior to testing, target dissolution was set at not less than 80% of drug released in 45 minutes. If 80% or more by weight of drug originally present in a tablet was in dissolved form 45 minutes after the start of the test, that tablet was deemed to have acceptable dissolution and to have “passed” the test. Results are shown in Table 2.

TABLE 2
Dissolution performance of valdecoxib tablets prepared
in Batches 1-11

| Tablet | | | |
|---|---|---|---|
| Batch | Starch Lot | % Dissolved at 45 min. | Passed? |
| 1 | A | 89 | Yes |
| 2 | B | 92 | Yes |
| 3 | C | 90 | Yes |
| 4 | D | 83 | Yes |
| 5 | E | 80 | Yes |
| 6 | F | 82 | Yes |
| 7 | G | 83 | Yes |
| 8 | H | 81 | Yes |
| 9 | I | 76 | No |
| 10 | J | 78 | No |
| 11 | K | 75 | No |

Data showing amount of drug dissolved in 45 minutes represent in each case an average of six tablets. The data in Table 2 indicate that tablets prepared using starch from lots A-H passed, but tablets prepared using starch from lots I-K did not pass, the dissolution test.

### Example 3

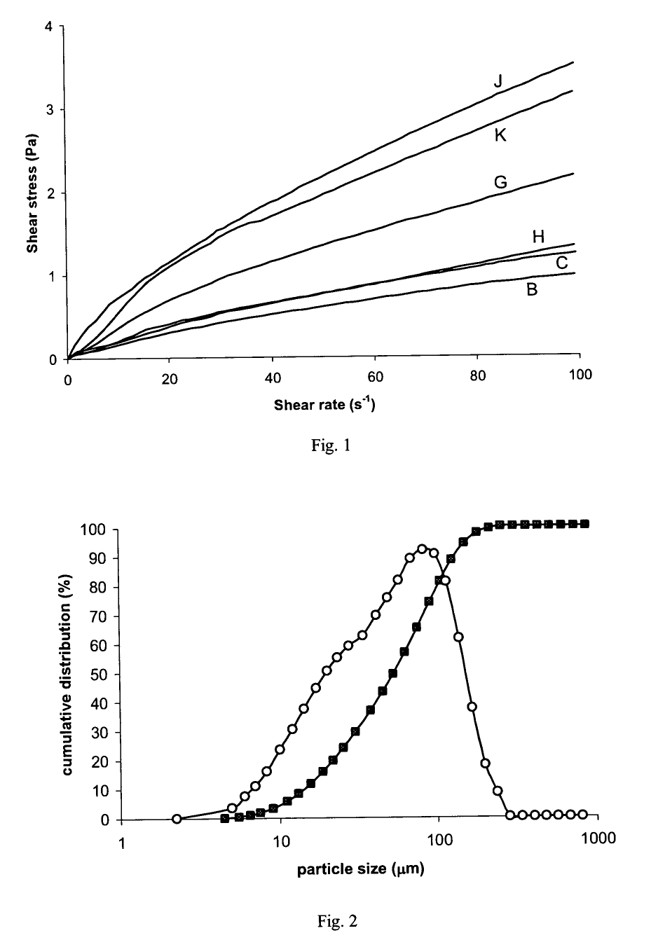

Additional samples of six of the Starch 1500 lots used in Example 1 were obtained. Samples of pregelatinized starch drawn from each of Lots B, C, G, H, J and K were placed in Test I described hereinabove to determine if these lots had “low viscosity” as defined herein. Test I was performed in triplicate and data were averaged. As shown in FIG. 1, pregelatinized starches sampled from four lots (Lots B, C, G and H) were determined to be of low viscosity, while pregelatinized starch sampled from the remaining two lots (Lots J and K) were determined not to be of low viscosity as defined herein.

Surprisingly and unexpectedly, these data indicate that tablets prepared with pregelatinized starch of low viscosity passed the in vitro dissolution test of Example 2 while otherwise identical tablets prepared with pregelatinized starch not meeting the low viscosity criterion did not pass the dissolution test.

Thus, if no selection of pregelatinized starch lot is made, tablets could pass or fail the dissolution test. Such inconsistency in dissolution perfromance is a serious problem in a commercial manufacturing setting. However, by selecting low viscosity pregelatinized starches in accordance with the invention, tablets can be prepared that pass the dissolution test every time. It is concluded that compositions of the invention exhibit improved in vitro dissolution rate consistency by comparison with compositions prepared using pregelatinized starch that has not been selected according to the present invention.

### Example 4

Additional samples of pregelatinized starch from Lots A-K as used in Example 1 were drawn from storage drums using a two-port Globe Pharma Unit Dose Powder Sampler equipped with 3 cm³ sample pockets. The sampler was approximately 33 cm in length. Samples were taken from three different positions in the drums, representing insertion of the sampler just below the surface of the starch (top), half-way into the drum (middle), and fully into the drum (bottom). Samples were transferred to a 45 cm³ bottle and capped until analyzed. Prior to weighing the samples for analysis, the bottles were turned horizontally and rotated 2-3 times. Approximately 500-600 mg of each sample (top, middle and bottom) was weighed in triplicate on weighing paper, using a top-loading balance.

Particle size distribution of each sample was determined using a Sympatec HELOS System Laser Light Diffraction Particle Size Apparatus, Model H0790 equipped with a RODOS+VIBRI dispersing system under the following instrument settings:

lens: R5 (focal length 500 mm);

pressure: 3.5 bar;

evacuation depression: 100 mbar;

feed-rate: 60%;

aperture: 4 mm;

cycle time: 100 ms;

time-out: 10 S.

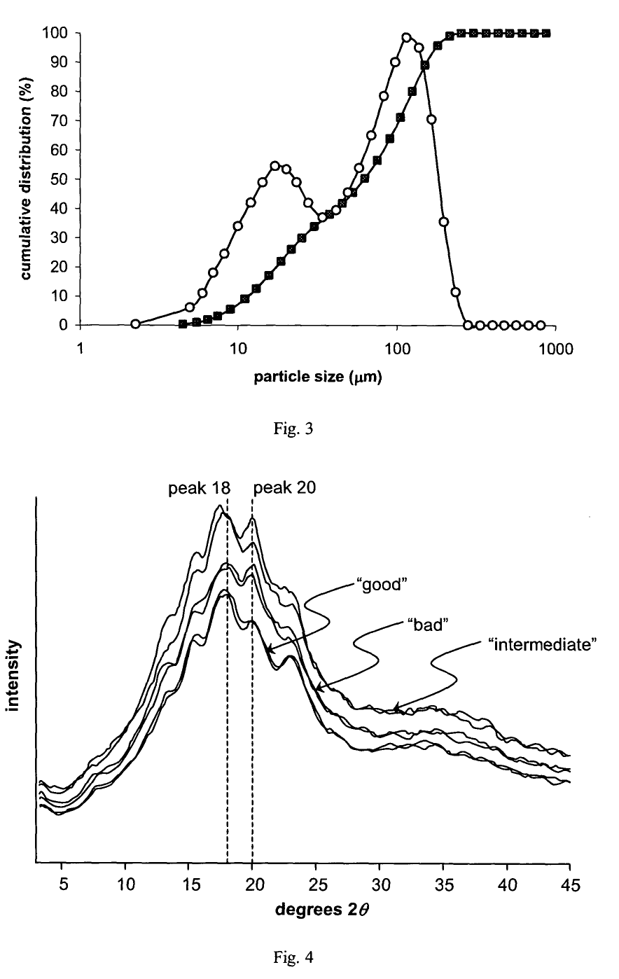

Samples from Lots A-H exhibited a bimodal particle size distribution while samples from Lots I-K exhibited a unimodal particle size distribution. Illustrative laser diffraction output representing starch that exhibited bimodal and unirnodal particle size distributions are shown in FIG. 2 (Lot H) and 3 (Lot K), respectively.

Results herein indicate that all tablets prepared with pregelatinized starch exhibiting a multimodal particle size distribution passed the in vitro dissolution test of Example 2 while all tablets prepared with pregelatinized starch exhibiting a unimodal particle size distribution failed the dissolution test. In the absence of a starch selection as provided by the present invention, tablets from approximately 27% of the batches prepared in Example 2 failed the established dissolution criteria; by contrast, using pregelatinized starch selected according to the present invention 100% of the batches passed the dissolution criteria. Therefore, compositions provided herein are advantageously capable of exhibiting improved in vitro dissolution rate consistency over compositions prepared using pregelatinized starch that is not selected according to the present invention.

### Example 5

Six pregelatinized starch lots were characterized by powder X-ray diffraction (PXRD). A sample (1.0 g) of each lot was packed tightly into a 50 mm diameter holder having 25 mm sample space, and was transferred to a Bruker D8 Advanced Diffractometer. Data were collected in the range from 2 to 70 degrees 2θ with a 0.02 degree step size and 1 second step time.

The six lots were selected to represent “good”, “intermediate” and “bad” lots (two lots from each category) based on a dissolution test of valdecoxib tablets prepared according to Example 1 with these lots, the dissolution test being similar to that of Example 2.

PXRD profiles are shown in FIG. 4. It was found that the ratio of intensity of peaks at about 18 and about 20 degrees 2θ (herein the “peak 18/peak 20 ratio”) was correlated with dissolution performance; specifically, “good” starches exhibited a high peak 18/peak 20 ratio and “bad” starches a low peak 18/peak 20 ratio. “Intermediate” starches exhibited a peak 18/peak 20 ratio intermediate between those of “good” and “bad” starches.

It is believed, without being bound by theory, that “good” starches (those providing tablets having favorable dissolution properties) are less highly processed than “bad” starches. PXRD of raw (unprocessed) starch has been found to exhibit a very high peak 18/peak 20 ratio (data not shown).

### Example 6

Samples of eleven pregelatinized starch lots were characterized by PXRD as in Example 5 and peak 18/peak 20 ratio was determined. Valdecoxib tablets were prepared by a wet granulation process as set forth in Example 1, and impeller power consumption profile was recorded. Surprisingly, a strong correlation was observed between the peak 18/peak 20 ratio and power consumption. This correlation became even stronger following removal of data for two outlying samples, as shown in FIG. 5. Starch lots having low peak 18/peak 20 ratio resulted in higher power consumption during wet granulation.

“Good” and “bad” starches can be qualitatively characterized in their properties and effects on granules (prepared by wet granulation as an intermediate in a tablet making process) and on finished tablets, as summarized in Table 3.

TABLE 3
Summary of properties and effects of
“good” and “bad” starches

| Material | Property or effect | “Good” | “Bad” |
|---|---|---|---|
| Starch | birefringence | less | more |
| | particle size distribution | bimodal | unimodal |
| | viscosity | lower | higher |
| | PXRD peak 18/peak 20 | higher | lower |
| | ratio | | |
| Granules | power consumption | lower | higher |
| | size | smaller | larger |
| | porosity | higher | lower |
| | surface area | greater | smaller |
| Tablets | dissolution | faster | slower |

What is claimed is:

1. An orally deliverable pharmaceutical composition comprising a drug of low water solubility and a pregelatinized starch having low viscosity and/or exhibiting a multimodal particle size distribution.

2. The composition of claim 1 that is in a form of a tablet or capsule.

3. The composition of claim 1 wherein the drug is a selective cyclooxygenase-2 inhibitory drug.

4. The composition of claim 3 wherein the selective cyclooxygenase-2 inhibitory drug is selected from the group consisting of celecoxib, deracoxib, valdecoxib, rofecoxib, etoricoxib, 2-(3,5-difluorophenyl)-3-[4-(methylsulfonyl)phenyl]-2-cyclopenten-1-one, 2-(3,4-difluorophenyl)-4-(3-hydroxy-3-methyl-1-butoxy)-5-[4-(methylsulfonyl)phenyl]-3-(2H)-pyridazinone, and pharmaceutically acceptable salts and prodrugs thereof.

5. The composition of claim 3 wherein the selective cyclooxygenase-2 inhibitory drug is valdecoxib.

6. The composition of claim 5 that is in the form of a tablet or capsule, wherein the valdecoxib is present in an amount of about 1 mg to about 100 mg.

7. The composition of claim 6 wherein the valdecoxib is present in an amount of about 5 mg to about 40 mg.

8. The composition of claim 5 wherein the valdecoxib has a D₉₀ particle size less than about 75 μm.

9. The composition of claim 1 wherein the pregelatinized starch exhibits a shear stress of not more than about 1 Pa at a shear rate of 20 s⁻¹.

10. The composition of claim 9 wherein the pregelatimzed starch further exhibits a shear stress of not more than about 2 Pa at a shear rate of 60 s⁻¹.

11. The composition of claim 10 wherein the pregelatinized starch further exhibits a shear stress of not more than about 3 Pa at a shear rate of 100 s⁻¹.

12. The composition of claim 1 wherein the pregelatinized starch exhibits a shear stress of not more than about 0.75 Pa at a shear rate of 20 s⁻¹.

13. The composition of claim 12 wherein the pregelatinized starch further exhibits a shear stress of not more than about 1.5 Pa at a shear rate of 60 s⁻¹.

14. The composition of claim 13 wherein the pregelatinized starch further exhibits a shear stress of not more than about 2.5 Pa at a shear rate of 100 s⁻¹.

15. The composition of claim 1 wherein the pregelatinized starch exhibits a shear stress of not more than about 0.5 Pa at a shear rate of 20 s⁻¹.

16. The composition of claim 15 wherein the pregelatinized starch further exhibits a shear stress of not more than about 1 Pa at a shear rate of 60 s⁻¹.

17. The composition of claim 16 wherein the pregelatinized starch further exhibits a shear stress of not more than about 1.5 Pa at a shear rate of 100 s⁻¹.

18. The composition of claim 1 wherein the pregelatinized starch exhibits a multimodal particle size distribution.

19. The composition of claim 1 wherein the pregelatinized starch exhibits a bimodal particle size distribution.

20. The composition of claim 1 wherein the starch is present in an amount of about 1% to about 50% by weight of the composition.

21. The composition of claim 1 wherein the starch is present in an amount of about 2.5% to about 30% by weight of the composition.

22. The composition of claim 1 that is in a form of a tablet, further comprising one or more diluents in an amount of about 5% to about 99%, one or more disintegrants in an amount of about 0.2% to about 30%, and one or more lubricants in an amount of about 0.1% to about 10%, by weight of the composition.

23. The composition of claim 1 that is in a form of a tablet, further comprising one or more excipients selected from the group consisting of lactose monohydrate, microcrystalline cellulose, croscarmellose sodium and magnesium stearate.

24. A process for preparing an orally deliverable pharmaceutical composition, the process comprising a step of selecting a pregelatinized starch having low viscosity and/or exhibiting a multimodal particle size profile, and a step of admixing the selected pregelatinized starch with a drug of low water solubility to provide an admixture.

25. The process of claim 24 wherein the drug is a selective cyclooxygenase-2 inhibitory drug.

26. The process of claim 25 wherein the selective cyclooxygenase-2 inhibitory drug is valdecoxib.

27. The process of claim 24, further comprising a step of wet granulating the admixture with one or more diluents, a step of drying the resulting granules, and a step of compressing the resulting dry granules to form a tablet.

28. A method of improving drug release rate consistency among pharmaceutical tablets prepared within a single manufacturing campaign, said tablets comprising pregelatinized starch and a drug having low water solubility, wherein the method comprises a step of selecting, for use in said tablets, a pregelatinized starch having low viscosity and/or exhibiting a multimodal particle size distribution.

29. A method of treating a medical condition or disorder in a subject where treatment with a cyclooxygenase-2 inhibitor is indicated, the method comprising orally administering to the subject a composition of claim 3 once or twice a day.
